(19) United States
(12) Patent Application Publication
(10) Pub. No.: US 2004/0235752 A1
(43) Pub. Date: Nov. 25, 2004

(54) 3-fluoro-pyrrolidines as antidiabetic agents

(76) Inventors: Gary Robert William Pitt, Hampshire (GB); David Michael Evans, Southampton (GB)

Correspondence Address: FOLEY AND LARDNER, SUITE 500, 3000 K STREET NW, WASHINGTON, DC, 20007, US

(21) Appl. No.: 10/481,798

(22) PCT Filed: Jun. 24, 2002
(86) PCT No.: PCT/GB02/02880

(30) Foreign Application Priority Data
Jun. 25, 2001 (GB) 0115517.5

(51) Int. Cl. A61K 38/04; A61K 31/4025; A61K 31/401

(52) U.S. Cl. 514/19; 514/423; 548/517; 548/528; 548/537

(57) ABSTRACT

Compounds according to general formula (1) and their pharmaceutically acceptable salts are new. The compounds are inhibitors of dipeptidyl peptidase IV or prodrugs thereof, and are useful in the treatment of, inter alia type 2 diabetes and impaired glucose tolerance. In the general formula A is F or H, one of R^{1A} and R^{1B} is H or CN and the other H, R² is H, alkyl, aralkyl or R⁵, R³ is H or a substituted aminoalkyl group and R⁴ is H or acyl.

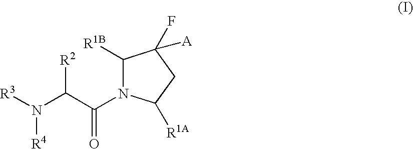

The present invention relates to novel compounds that are inhibitors of dipeptidyl peptidase IV or prodrugs thereof. The compounds are useful in the treatment of, inter alia, type 2 diabetes and impaired glucose tolerance.

### BACKGROUND

The enzyme dipeptidyl peptidase IV, herein abbreviated DP-IV (and elsewhere as DAP-IV or DPP-IV) and also known by the classification EC.3.4.14.5, is a serine protease that cleaves the N-terminal dipeptide from peptides that begin with the sequence H-Xaa-Pro (where Xaa is any amino acid, although preferably a lipophilic one, and Pro is proline). It will also accept as substrates peptides that begin with the sequence H-Xaa-Ala (where Ala is alanine). DP-IV was first identified as a membrane-bound protein. More recently a soluble form has been identified.

Initial interest in DP-IV focussed on its role in the activation of T lymphocytes. DP-IV is identical to the T cell protein CD26. It was proposed that inhibitors of DP-IV would be capable of modulating T cell responsiveness, and so could be developed as novel Immunomodulators. It was further suggested that CD26 was a necessary co-receptor for HIV, and thus that DP-IV inhibitors could be useful in the treatment of AIDS.

Attention was given to the role of DP-IV outside the immune system. It was recognised that DP-IV has a key role in the degradation of several peptide hormones, including growth hormone releasing hormone (GHRH) and glucagon-like peptide-1 and -2 (GLP-1 and GLP-2). Since GLP-1 is known to have a potentiating effect on the action of insulin in the control of post-prandial blood glucose levels it is clear that DP-IV inhibitors might also be usefully employed in the treatment of type II diabetes and impaired glucose tolerance. At least two DP-IV inhibitors are currently undergoing clinical trials to explore this possibility.

Several groups have disclosed inhibitors of DP-IV. While some leads have been found from random screening programs, the majority of the work in this field has been directed towards the investigation of substrate analogs. Inhibitors of DP-IV that are substrate analogs are disclosed in, for example, U.S. Pat. No. 5,462,928, U.S. Pat. No. 5,543,396, WO95/15309 (equivalent to U.S. Pat. No. 5,939,560 and EP 0731789), WO98/19998 (equivalent to U.S. Pat. No. 6,011,155), WO99/46272 and WO99/61431. The most potent inhibitors are aminoacyl pyrrolidine boronic acids, but these are unstable and tend to cyclise, while the more stable pyrrolidine and thiazolidine derivatives have a lower affinity for the enzyme and so would require large doses in a clinical situation. Pyrrolidine nitrites appear to offer a good compromise since they have both a high affinity for the enzyme and a reasonably long half-life in solution as the free base. There remains, however, a need for inhibitors of DP-IV with improved properties.

### SUMMARY OF THE INVENTION

The present invention relates to a series of inhibitors of DP-IV with improved affinity for the enzyme and prodrugs thereto. The compounds can be used for the treatment of a number of human diseases, including impaired glucose tolerance and type II diabetes. Accordingly, the invention further relates to the use of the compounds in the preparation of pharmaceutical compositions, to such compositions per se, and to the use of such compositions in human therapy. The compounds of the invention are described by general formula 1.

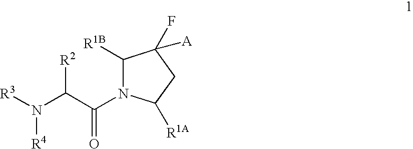

In this general formula A is F or H; one of R^{1A} and R^{1B} is selected from H and CN and the other is H; R² is selected from H, C₁-C₈ alkyl, optionally substituted phenyl, optionally substituted benzyl and R⁵; R³ is selected from H, R⁶OCO, H₂NCH(R⁷)CO, H₂NCH(R⁸)CONHCH(R⁹)CO, and a group according to general formula 2;

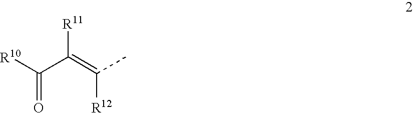

R⁴ is selected from H, C₁-C₈ alkyl, adamantyl, adamantylmethyl, adamantylethyl and Het-NH(CH₂)ₐ; or R² and R⁴ together constitute a chain of three or four methylene groups so as to form, together with the atoms to which they are attached, a pyrrolidine or piperidine ring, which ring may further be fused with a benzenoid ring; R⁵ is selected from CH₂R¹³, CH₂CH₂R¹³ and C(R¹⁴)(R¹⁵)—X¹—R¹⁶; R⁶ is selected from C₁-C₆ alkyl, optionally substituted phenyl, optionally substituted benzyl and R¹⁷CO₂C(R¹⁸)(R¹⁹): R⁷, R⁸ and R⁹ are each independently selected from the side chains of the proteinaceous amino acids; R¹⁰ is selected from C₁-C₈ alkyl, phenyl and O—(C₁-C₈ alkyl); R¹¹ is selected from H and C₁-C₈ alkyl; R¹² is selected from H, C₁-C₈ alkyl and phenyl; R¹³ is selected from CO—N(R²⁰)(R²¹), N(R²²)—C(═X²)R²³ and N(R²²)(R²⁴); R¹⁴ and R¹⁵ are independently selected from H and methyl, or together are —(CH₂)_{z}—; R¹⁶ is selected from C₁-C₈ alkyl, optionally substituted phenyl, optionally substituted benzyl and —(CH₂)_{b}—R¹³; R¹⁷ is selected from H and C₁-C₈ alkyl; R¹⁶ and R¹⁹ are independently selected from H and C₁-C₈ alkyl, or together are —(CH₂)_{y}—; R²⁰ and R²¹ are independently selected from H, C₁-C₈ alkyl, optionally substituted phenyl, optionally substituted phenylalkyl, Het and —(CH₂)_{c}Het, or R²⁰ and R²¹ together constitute a chain of four or five methylene groups so as to form, together with the nitrogen atom to which they are attached, a pyrrolidine or piperidine ring, which ring may further be fused with a benzenoid ring; R²² is selected from H and methyl; R²³ is selected from R²⁵, O—R²⁵ and N(R²⁶)(R²⁷); R²⁴ is selected from optionally substituted phenyl, Het and —CH₂-Het, R²⁵ is selected from C₁-C₈ alkyl, optionally substituted phenyl, optionally substituted phenylalkyl, Het and —CH₂)_{c}Het; R²⁶ and R²⁷ are independently selected from H, C₁-C₈ alkyl, optionally substituted phenyl, optionally substituted phenylalkyl, Het and —(CH₂)_{c}Het, or R²⁶ and R²⁷ together constitute a chain of four or five methylene groups so as to form, together with the nitrogen atom to which they are attached, a pyrrolidine or piperidine ring, which ring may further be fused with a benzenoid ring; Het is an optionally substituted aromatic nitrogen-containing heterocycle or benz-fused analogue thereof; X¹ is selected from —O—, —S— and —CH₂—; X² is selected from O and S; a is 2 or 3; b is 1, 2 or 3; c is 1 or 2; and y and z are 2, 3 or 4.

### DETAILED DESCRIPTION OF THE INVENTION

In a first aspect, the present invention comprises a series of novel compounds that are inhibitors of the enzyme DP-IV or prodrugs thereof and are useful for the treatment of certain human diseases. The compounds are described by general formula 1.

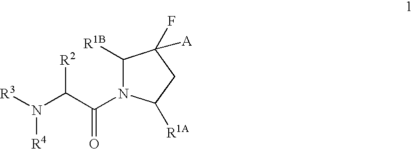

In general formula 1, the atom A may be either hydrogen (H) or fluorine (F). Preferably it is F. One of R^{1A} and R^{1B} may be a nitrile group (CN) and the other H. Alternatively both R^{1A} and R^{1B} may be H. In one preferred embodiment of the invention both R^{1A} and R^{1B} are H. In another preferred embodiment of the invention R^{1A} is CN and R¹⁸ is H.

In one particularly preferred embodiment, A is F and both R^{1A} and R^{1B} are H. In another particularly preferred embodiment A is F, R^{1A} is CN and R^{1B} is H.

In one embodiment of the present invention R² is a group selected from H, C₁-C₈ alkyl groups, an optionally substituted phenyl residue, an optionally substituted benzyl group and groups according to R⁵. Suitable optional substituents on the phenyl residue or the benzyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl residue or benzyl group may have up to three substituents, which may all be the same or may be different. In this embodiment, R³ is a group selected from H, C₁-C₈ alkyl groups, adamantyl, adamantylmethyl, adamantylethyl and a group according to Het-NH(CH₂)ₐ, where a is 2 or 3.

In a second embodiment of the present invention, R² and R³ together constitute a chain of three or four methylene groups so as to form, together with the atoms to which they are attached, a pyrrolidine or piperidine ring. This ring may further be fused with a benzenoid ring so as to form an indoline, isoindoline, tetrahydroquinoline or tetrahydroisoquinoline moiety.

For those compounds according to the present invention that are direct inhibitors of DP-IV, R⁴ is H. For those compounds according to the present invention that are prodrugs of these direct inhibitors, R⁴ is selected from a group according to R⁶OCO, a group according to H₂NCH(R⁷)CO, a group according to H₂NCH(R⁸)CONHCH(R⁹)CO, and a group according to general formula 2.

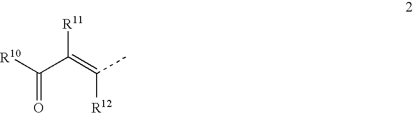

These prodrugs are converted into the corresponding direct inhibitors of DP-IV after administration to the patient.

The group R⁵ is selected from a group according to CH₂R¹³, a group according to CH₂CH₂R¹³ and a group according to C(R¹⁴)(R¹⁵)—X¹—R¹⁶, where X¹ is selected from —O—, —S— and —CH₂—.

The group R⁶ is selected from C₁-C₈ alkyl groups, an optionally substituted phenyl or benzyl group and a group according to R¹⁷CO₂C(R¹⁸)(R¹⁹). Suitable substituents on the phenyl or benzyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl or benzyl group may have up to two substituents, which may be the same or different.

The groups R⁷, R⁸ and R⁹ are each independently selected from the side chains of the proteinaceous amino acids. These amino acids and their side chains are enumerated in the Table below.

| | |
|---|---|
| Alanine | —CH₃ |
| Arginine | —(CH₂)₃NHC(═NH)NH₂ |
| Asparagine | —CH₂CONH₂ |
| Aspartic acid | —CH₂CO₂H |
| Cysteine | —CH₂SH |
| Glycine | —H |
| Glutamic acid | —(CH₂)₂CO₂H |
| Glutamine | —(CH₂)₂CONH₂ |
| Histidine | —CH₂C3H3N2 |
| Isoleucine | —CH(CH₃)CH₂CH₃ |
| Leucine | —CH₂CH(CH₃)₂ |
| Lysine | —(CH₂)₄NH₂ |
| Methionine | —(CH₂)₂SCH₃ |
| Phenylalanine | —CH₂C₆H₅ |
| Serine | —CH₂OH |
| Threonine | —CH(CH₃)OH |
| Tryptophan | —CH₂C₈H₆N |
| Tyrosine | —CH₂C₆H₄OH |
| Valine | —CH(CH₃)₂ |

In general formula 2, the group R¹⁰ is selected from C₁-C₈ alkyl groups, phenyl and O—(C₁-C₈ alkyl) groups, the group R¹¹ is selected from H and C₁-C₈ alkyl groups, and the group R¹² is selected from H, C₁-C₈ alkyl groups and phenyl.

The group R¹³ is selected from a group according to CO—N(R²⁰(R²¹), a group according to N(R²²)—C(═X²)R²³, where X² is selected from O and S, and a group according to N(R²²)(R²⁴).

The groups R¹⁴ and R¹⁵ are independently selected from H and methyl, or together are —(CH₂)_{z}—, where z is 2, 3 or 4, so as to form, together with the carbon atom to which they are attached, a cyclopropane, cyclobutane or cyclopentane ring.

The group R¹⁶ is selected from C₁-C⁸ alkyl groups, an optionally substituted phenyl. group, an optionally substituted benzyl group and groups according to —(CH₂)_{b}—R¹³, where b is 1, 2 or 3. Suitable substituents on the phenyl or benzyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl or benzyl group may. have up to two substituents, which may be the same or different.

The group R¹⁷ is selected from H and C₁-C₈ alkyl groups. The groups R¹⁸ and R¹⁹ are independently selected from H and C₁-C₈ alkyl groups, or together are —(CH₂)_{y}—, where y is 2, 3 or 4, so as to form, together with the carbon atom to which they are attached, a cyclopropane, cyclobutane or cyclopentane ring

The groups R²⁰ and R²¹ may independently be selected from H, C₁-C₈ alkyl groups, an optionally substituted phenyl group, an optionally substituted phenylalkyl group, a group according to Het and a group according to —(CH₂)_{c}Het, where c is 1 or 2. Suitable substituents on the phenyl or phenylalkyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl or phenylalkyl group may have up to two substituents, which may be the same or different. Alternatively, the groups R²⁰ and R²¹ may together constitute a chain of four or five methylene groups so as to form, together with the, nitrogen atom to which they are attached, a pyrrolidine or piperidine ring, which ring may further be fused with a benzenoid ring so as to form an indoline, isoindoline, tetrahydroquinoline or tetrahydroisoquinoline moiety.

The group R²² is selected from H and methyl. The group R²³ is selected from a group according to R²⁵, a group according to O—R²⁵ and a group according to N(R²⁶)(R²⁷). The group R²⁴ is selected from an optionally substituted phenyl group, a group according to Het and a group according to —CH₂-Het. Suitable substituents on the phenyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl group may have up to two substituents, which may be the same or different

The group R²⁵ is selected from C₁-C₈ alkyl groups, an optionally substituted phenyl group, an optionally substituted phenylalkyl group, a group according to Het and a group according to —(CH₂)_{c}Het. Suitable substituents on the phenyl or phenylalkyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl or phenylalkyl group may have up to two substituents, which may be the same or different

The groups R²⁶ and R²⁷ may independently be selected from H, C₁-C₈ alkyl groups, an optionally substituted phenyl group, an optionally substituted phenylalkyl group, a group according to Het and a group according to —(CH₂)_{c}Het. Suitable substituents on the phenyl or phenylalkyl group are lower alkyl groups, lower alkyloxy groups, halogen atoms selected from fluorine and chlorine atoms, hydroxyl groups, amino groups selected from NH₂, NH-(lower alkyl) and N(lower alkyl)₂, nitrile groups, nitro groups, CO₂H, CO₂-(lower alkyl), CONH₂, CONH-(lower alkyl) and CON(lower alkyl)₂. The phenyl or phenylalkyl group may have up to two substituents, which may be the same or different. Alternatively R²⁶ and R²⁷ may together constitute a chain of four or five methylene groups so as to form, together with the nitrogen atom to which they are attached, a pyrrolidine or piperidine ring, which ring may further be fused with a benzenoid ring so as to form an indoline, isoindoline, tetrahydroquinoline or tetrahydroisoquinoline moiety.

Het is an aromatic nitrogen-containing heterocyclic group selected from pyridyl, pyridazinyl, pyrimidinyl, pyrazinyl, imidazolyl, pyrazolyl, thiazolyl, isothiazolyl, oxazolyl, isoxazolyl and benz-fused analogues of these, such as for example quinolinyl, Isoquinollnyl, quinoxallnyl, benzimidazolyl and the like, all of which may optionally be substituted on one or more carbon atoms, and where the substituents are selected from lower alkyl, hydroxy, lower alkyloxy, amino, lower alkylamino, di(lower alkyl)amino, fluoro, chloro, bromo, trifluoromethyl, nitro, cyano, carboxy and lower alkyloxycarbonyl groups;

In the context of the present document, the term “alkyl group”, either by itself or in combinations such as “alkyloxy”, includes linear, branched and cyclic saturated hydrocarbon groups. Examples of C₁-C₈ alkyl groups include methyl, ethyl, propyl, n-octyl, 2,2,4-trimethylpentyl and bicyclo[2.2.2]octyl groups. Lower alkyl groups are alkyl groups with up to four carbon atoms, i.e. C₁-C₄ alkyl groups such as methyl, ethyl, propyl, isopropyl, cyclopropyl, butyl, isobutyl, tert-butyl and cyclobutyl groups. The term “phenylalkyl group” includes lower alkyl groups with a phenyl substituent. Examples of phenylalkyl groups include benzyl, phenethyl, α-methylbenzyl and 4-phenylbutyl groups.

The compounds of general formula 1 may have one or more stereogenic centres and so can exhibit optical isomerism. All such isomers, including enantiomers, diastereomers and epimers are included within the scope of the invention. Furthermore, the invention includes such compounds as single isomers and as mixtures, including racemates. Certain compounds according to general formula 1, including those with a heteroaryl group which carries a hydroxy or amino substituent, can exist as tautomers. These tautomers, either separately or as mixtures, are also considered to be within the scope of the invention.

The compounds according to general formula 1 wherein R⁴ is H have at least one basic functional group. They can therefore form addition salts with acids. Other compounds according to general formula 1 wherein R⁴ is not H may also have a basic functional group and so be able to form addition salts. Insofar as these addition salts are formed with pharmaceutically acceptable acids, they are included within the scope of the invention. Examples of suitable acids include acetic acid, trifluoroacetic acid, citric acid, fumaric acid, benzoic acid, pamoic acid, methanesulphonic acid, hydrochloric acid, nitric acid, sulphuric acid, phosphoric acid and the like.

Certain compounds according to general formula 1 have an acidic group and so are able to form salts with bases. Examples of such salts include the sodium, potassium and calcium salts, which are formed by the reaction of the acid with the corresponding metal hydroxide, oxide, carbonate or bicarbonate. Similarly, tetra-alkyl ammonium salts may be formed by the reaction of the acid with a tetra-alkyl ammonium hydroxide. Primary, secondary and tertiary amines, such as triethylamine, can form addition salts with the acid. A particular case of this would be an internal addition salt formed between an acidic group and the primary amine group of the same molecule, which is also called a zwitterion. Insofar as they are pharmaceutically acceptable, all these salts are included within the scope of the invention.

It is generally preferred that R² and R³ should not both be H. In embodiments of the invention wherein R² is H, R³ is preferably selected from adamantyl, adamantylmethyl, adamantylethyl and groups according to Het-(CH₂)ₐ. More preferably it is a group according to Het-(CH₂)ₐ, and most preferably it is such a group wherein a is 2 and Het is a 5-substituted-2-pyridyl moiety.

More preferred are those embodiments of the invention wherein R³ is H and R²is selected from C₁-C₈ alkyl groups, an optionally substituted phenyl residue, an optionally substituted benzyl group and groups according to R⁵.

One particularly preferred embodiment of the invention is a compound wherein R³ is H and R² is a C₁-C⁸ alkyl group.

Another particularly preferred embodiment is a compound wherein R³ is H and R² is a group according to R⁵. More preferred still are those compounds wherein R⁵ is either CH₂CH₂R¹³ or C(R¹⁴)(R¹⁵)—X¹—R¹⁶. Preferred compounds with R⁵ as CH₂CH₂R¹³ are those wherein R¹³ is CO—N(R²⁰)(R²¹). Preferred compounds with R⁵ as C(R¹⁴)(R¹⁵)—X¹—R¹⁶ are those wherein R¹⁴ and R¹⁵ are either H or methyl and R¹⁶ is —(CH₂)_{b}—R³, particularly those wherein R¹⁴ and R¹⁵ are both H, X¹ is CH₂ and b is 1 or 2, more particularly those wherein R¹³ is either N(R²²)—C(═X²)R²³ or N(R²²)(R²⁴), more particularly still those wherein R¹³ is N(R²²)—C(═X₂)R²³, R²² is H and X² is O, and most particularly those wherein R²³ is Het.

Another preferred embodiment of the present invention is a compound according to general formula 1 wherein R² is other than H and the absolute stereochemistry is as shown in general formula 3. In the conventional system of nomenclature this is the ‘S’ configuration, except where R² is R⁵, R⁵ is C(R¹⁴)(R¹⁵)—X¹—R¹⁶ and X¹ is S, in which case it is the ‘R’ configuration.

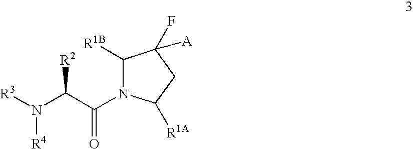

Another preferred embodiment of the present invention is a compound according to general formula 1 wherein R^{1A} is CN, R^{1B} is H and the absolute stereochemistry is as shown in general formula 4. In. the conventional system of nomenclature this is the ‘S’ configuration.

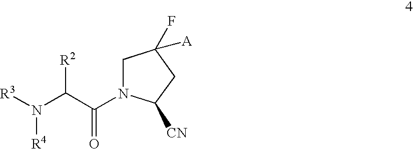

Another preferred embodiment of the present invention is a compound according to general formula 1 wherein R^{1A} is H, R^{1B} is CN and the absolute stereochemistry is as shown in general formula 5. In the conventional system of nomenclature this is the ‘R’ configuration.

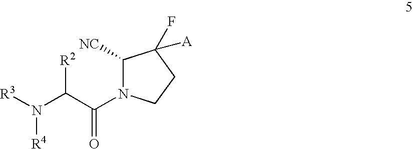

The compounds according to general formula 1 can be prepared using conventional synthetic methods.

Compounds wherein R⁴ is other than H are generally accessible from the corresponding compounds wherein R⁴ is H. When R⁴ is R⁶OCO— the desired compound can usually be prepared by the reaction of the amine functional group with a suitable carbonic acid derivative.

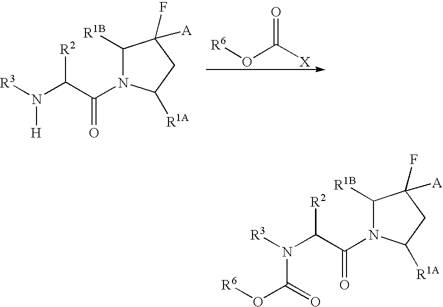

Here X is a leaving group such as a chlorine atom (Cl) or a para-nitrophenoxy group (O₂NC₆H₄O)

Compounds wherein R⁴ is a group according to general formula 2 can be prepared by the reaction of the amine functional group with a 1,3-dicarbonyl compound such as a 1,3-diketone or a β-ketoester.

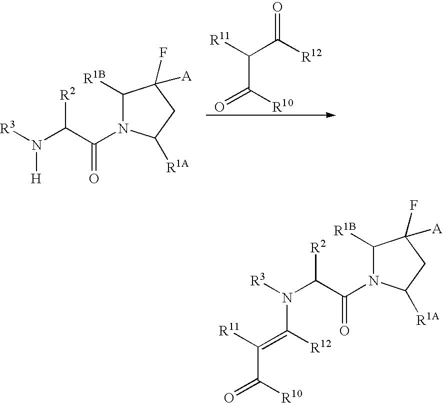

Compounds wherein R⁴ is an amino acyl group H₂NCH(R⁷)CO— can as be prepared by the conventional methods of peptide synthesis.

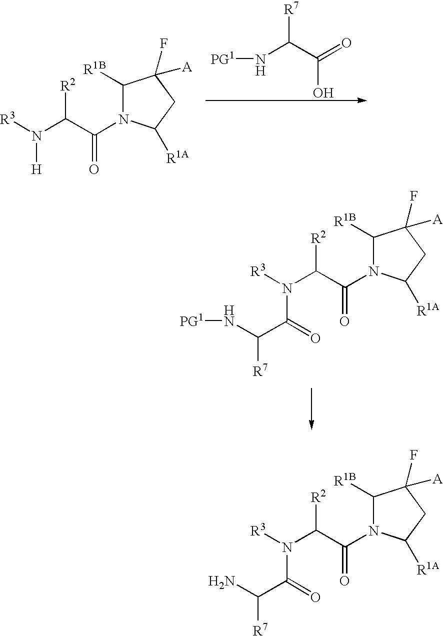

In a first step, the amine is reacted with a protected amino acid in the presence of a coupling agent. PG¹ is a protecting group such as tert-butyloxycarbonyl (BOC). benzyloxycarbonyl (Z) or 9-fluorenylmethyloxycarbonyl (Fmoc). The use of such groups is well known in the art. Where R⁷ has a reactive functional group such as an amine or a carboxylic acid, this group will also be protected. In a second step the protecting group is removed.

Compounds wherein R⁴ is a group H₂NCH(R⁸)CONHCH(R⁹)CO— can also be prepared by the conventional methods of peptide synthesis.

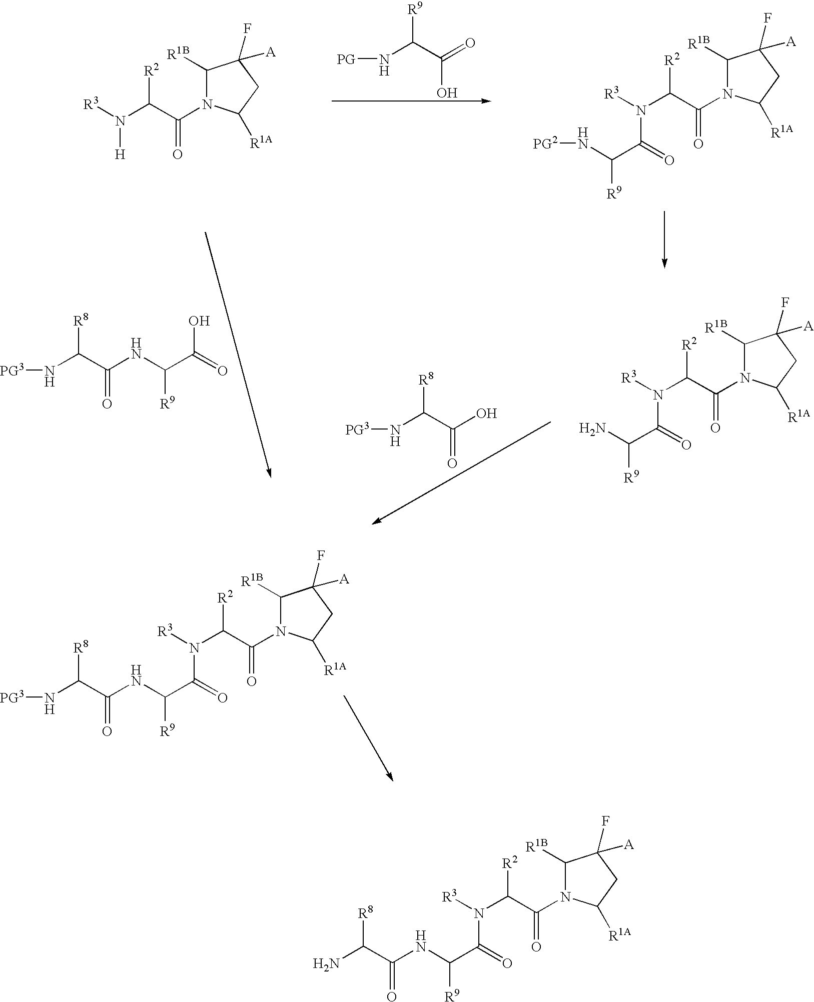

Here again, PG² and PG³ are protecting groups. The side chains R⁸ and R⁹ may also have protecting groups if necessary. The target compound may be assembled in a stepwise process or directly by coupling of a dipeptide fragment.

The most direct route to the compounds of the invention wherein R⁴ is H is by the coupling of an appropriately functionalised and protected amino acid and a pyrrolidine derivatives.

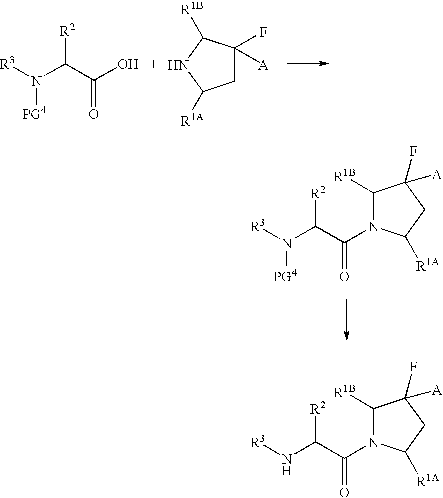

In some circumstances, such as when a large number of different compounds are to be made, it may be more convenient to prepare a compound that can serve as a common intermediate. For example, when a number of compounds are required wherein R² is CH₂CH₂CON(R²⁰)(R²¹), it is convenient to prepare a common intermediate with R² being CH₂CH₂CO₂H and derivatise this by reaction with different amines.

The pyrrolidine derivatives are either known compounds or can be prepared by simple modification of published synthetic routes. These preparations are described in detail in the Examples.

In a second aspect, the present invention comprises a pharmaceutical composition for human therapeutic use. The composition is characterised in that it has, as an active agent, at least one of the compounds described above. Such a composition is useful in the treatment of human diseases. The composition will generally Include one or more additional components selected from pharmaceutically acceptable excipients and pharmaceutically active agents other than those of the present invention.

The composition may be presented as a solid or liquid formulation, depending on the intended route of administration. Examples of solid formulations include pills, tablets, capsules and powders for oral administration, suppositories for rectal or vaginal administration, powders for nasal or pulmonary administration, and patches for transdermal or transmucosal (such as buccal) administration. Examples of liquid formulations include solutions and suspensions for intravenous, subcutaneous or intramuscular injection and oral, nasal or pulmonary administration. A particularly preferred presentation is a tablet for oral administration. Another preferred presentation, particularly for emergency and critical care, is a sterile solution for intravenous injection.

The composition comprises at least one compound according to the preceding description. The composition may contain more than one such compound, but in general it is preferred that it should comprise only one. The amount of the compound used in the composition will be such that the total daily dose of the active agent can be administered n one to four convenient dose units. For example, the composition can be a tablet containing an amount of compound equal to the total daily dose necessary, said tablet to be taken once per day. Alternatively, the tablet can contain half (or one third, or one quarter) of the daily dose, to be taken twice (or three or four times) per day. Such a tablet can also be scored to facilitate divided dosing, so that, for example, a tablet comprising a full daily dose can be broken into half and administered in two portions. Preferably, a tablet or other unit dosage form will contain between 0.1 mg and 1 g of active compound. More preferably, it will contain between 1 mg and 250 mg.

The composition will generally include one or more excipients selected from those that are recognised as being pharmaceutically acceptable. Suitable excipients include, but are not limited to, bulking agents, binding agents, diluents, solvents, preservatives and flavouring agents. Agents that modify the release characteristics of the composition, such as polymers that selectively dissolve in the intestine (“enteric coatings”) are also considered in the context of the present invention, to be suitable excipients.

The composition may comprise, in addition to the compound of the invention, a second pharmaceutically active agent. For example, the composition may include an anti-diabetic agent, a growth-promoting agent, an anti-inflammatory agent or an antiviral agent. However, it is generally preferred that the composition comprise only one active agent.

In a third aspect, the invention comprises a use for the compounds and compositions described above for the treatment of human diseases. This aspect can equally be considered to comprise a method of treatment for such diseases. The diseases susceptible to treatment are those wherein an inhibition of DP-IV or CD26 results in a clinical benefit either directly or indirectly. Direct effects include the blockade of T lymphocyte activation. Indirect effects include the potentiation of peptide hormone activity by preventing the degradation of these hormones. Examples of diseases include, but are not limited to, auto-immune and inflammatory diseases such as inflammatory bowel disease and rheumatoid arthritis, growth hormone deficiency leading to short stature, polycystic ovary syndrome, impaired glucose tolerance and type 2 diabetes. Particularly preferred is the use of the compounds and compositions for the treatment of impaired glucose tolerance and type 2 diabetes, and equally a method of treatment of these diseases by the administration of an effective amount of a compound or composition as previously described.

The precise details of the treatment, including the dosing regimen, will be established by the attending physician taking into account the general profile of the patient and the severity of the disease. For diseases such as inflammatory bowel disease that have acute phases of active disease separated by quiescent periods, the physician may select a relatively high dose during the acute phase and a lower maintenance dose for the quiescent period. For chronic diseases such as type 2 diabetes and impaired glucose tolerance, the dosing may need to be maintained at the same level for an extended period. A dosing schedule of one to four tablets per day, each comprising between 0.1 mg and 1 g (and preferably between 1 mg and 250 mg) of active compound might be typical in such a case.

The invention is further illustrated with the following non-limiting Examples.

### EXAMPLES

### Example 1

### (2S)-4,4-Difluoro-1-[N^{ω}-(pyrazinyl-2-carbonyl)-L-ornithinyl]-pyrrolidine-2-carbonitrile Trifluoroacetate

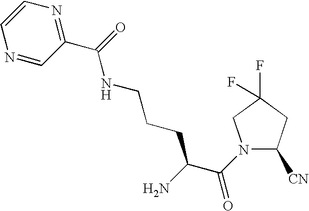

1A. Methyl (2S)-N-(tert-butyloxycarbonyl)-4-pyrrolidone-2-carboxylate

N-(tert-Butyloxycarbonyl)-L-4-trans-hydroxyproline methyl ester (2.5 g, 10.2 mmol) was dissolved in, CH₂Cl₂ (70 ml). Dess-Martin periodinane (5.09, 12.1 mmol) was added and the mixture was stirred for 3 hours at room temperature. The solvent was removed in) vacuo and the residue was taken up in ethyl acetate (300 ml). The solution was washed with sat. NaHCO₃, water and brine, dried (Na₂SO₄) and evaporated in vacuo to give a colourless oil. The residue was purified by flash chromatography (eluant: 10% ethyl acetate, 90% pet. ether 60-80) to give a colourless oil identified as methyl (2S)-N-(tert-butyloxycarbonyl)-4-pyrrolidone-2-carboxylate (2.4 g, 9.7 mmol, 95%).

1B. Methyl (2S)-N-(tert-butyloxycarbonyl)-4,4-difluoropyrrolidine-2-carboxylate

Methyl (2S)-N-(tert-butyloxycarbonyl)-4-pyrrolidone-2-carboxylate (2.3 g, 9.3 mol) was dissolved in CH₂Cl₂ (70 ml). (Diethylamino)sulphur trifluoride (4.5 g, 27.9 mmol) was added to this solution at 0° C. and the mixture was stirred for 18 hours at 0° C. to room temperature. The reaction mixture was carefully poured into sat. NaHCO₃ (100 ml) and the mixture was stirred for 15 min then extracted with CH₂Cl₂. The organic extract was washed with water and brine, dried (Na₂SO₄) and evaporated in vacuo to give an orange oil. The residue was purified by flash chromatography (eluant: 10% ethyl acetate, 90% pet. ether 60-80) to give a colourless oil identified as methyl (2S)-N-(tert-butyloxycarbonyl)-4,4-difluoropyrrolidine-2-carboxylate (2.4g, 8.9 mmol, 96%).

1C. (2S)-N-(tert-Butyloxycarbonyl)-4,4-difluoropyrrolidine-2-carboxylic Acid

Methyl (2S)-N-(tert-butyloxycarbonyl)-4,4-difluoropyrrolidine-2-carboxylate (2.2 g, 8.3 mmol) was dissolved in THF (100 ml). Aqueous lithium hydroxide (1M, 10.6 ml, 10.6 mmol) was added. The mixture was stirred for 3 hours at room temperature then diluted with ethyl acetate (150 ml), washed with 1M HCl, water and brine, dried (Na₂SO₄) and evaporated in vacuo to give an orange oil. The residue was purified by flash chromatography (eluant: 95% chloroform, 4% methanol, 1% acetic acid) to give an orange oil identified as (2S)-N-(tert-butyloxycarbonyl)-4,4-difluoropyrrolidine-2-carboxylic acid (2.1 g, 8.3 mmol, 100%).

1D. (2S)-N-(tert-Butyloxycarbonyl)-4,4-difluoropyrrolidine-2-carboxamide

(2S)-N-(tert-Butyloxycarbonyl)-4,4-difluoropyrrolidine-2-carboxylic acid (1.0 g, 4.0 mmol) was dissolved in CH₂Cl₂/DMF (9:1, 50 ml). To this solution at 0° C. was added 1-hydroxybenzotriazole hydrate (1.1 g, 8.1 mmol) and water-soluble carbodiimide (960 mg, 4.8 mmol). The mixture was stirred for 1 hour at 0° C. then ammonia (35%, 5 ml) was added. The mixture was stirred for 18 h at 0° C. to room temperature then the solvent was removed in vacuo and the residue was taken up in ethyl acetate (200 ml). The solution was washed with 0.3 M KHSO₄, sat. NaHCO₃, water and brine, dried (Na₂SO₄) and evaporated in vacuo to give a yellow oil. The residue was purified by flash chromatography (eluant: 85% ethyl acetate, 15% pet. ether 60-80) to give a colourless oil identified as (2S)-N-(tert-butyloxycarbonyl)-4,4-difluoropyrrolidine-2-carboxamide (945 mg, 3.8 mmol, 95%).

1E. (2S)-1-N^{α}-(tert-Butyloxycarbonyl)-N^{ω}-(pyrazinyl-2-carbonyl)-L-ornithinyl)-4,4-difluoropyrrolidine-2-carboxamide

(2S)-N-(tert-Butyloxycarbonyl)-4,4-difluoropyrrolidine-2-carboxamide (130 mg, 0.54 mmol) was dissolved in 4M HCl/dioxan (30 ml). The solution was stirred for 1 hour at room temperature then the solvent was removed in vacuo and the residue was dissolved in CH₂Cl₂/DMF (9:1, 20 ml). To this solution at 0° C. was added N^{α}-(tert-butyloxycarbonyl)-N^{ω}-(pyrazinyl-2-carbonyl)-L-ornithine (180 mg, 0.53 mmol), 1-hydroxybenzotriazole hydrate (9 mg, 0.67 mmol) and water-soluble carbodiimide (136 mg, 0.65 mmol). The mixture was stirred for 15 mins at 0° C. then the pH was adjusted to pH8 with N-methylmorpholine. The mixture was stirred for 18 h at 0° C. to room temperature then the solvent was removed in vacuo and the residue was taken up in ethyl acetate (70 ml). The solution was washed with 0.3M KHSO₄, sat. NaHCO₃, water and brine, dried (Na₂SO₄) and evaporated in vacuo to give a yellow oil. The residue was purified by flash chromatography (eluant: 92% chloroform, 8% methanol) to give a white solid identified as (2S)-1[N^{α}-(tert-butyloxycarbonyl)-N^{ω}-(pyrazinyl-2-carbonyl)-L-ornithinyl]-4,4-difluoropyrrolidine-2-carboxamide (195 mg, 0.41 mmol, 77%).

1F. (2S)-1-[N^{α}-(tert-Butyloxycarbonyl)-N^{ω}(pyrazinyl-2-carbonyl)-L-ornithinyl]-4,4-difluoropyrrolidine-2-carbonitrile

(2S)-[N^{α}(tert-Butyloxycarbonyl)-N^{ω}-(pyrazinyl-2-carbonyl)-L-ornithinyl]-4,4-difluoropyrrolidine-2-carboxamide (175 mg, 0.37 mmol) was dissolved in dry THF (30 ml). This solution was cooled to 0° C. then triethylamine (75 mg, 0.75 mmol) was added followed by trifluoroacetic anhydride (190 mg, 0.9 mmol). The mixture was stirred for 5 min then the pH was adjusted to pH9 with triethylamine. The mixture was stirred for a further 30 min then diluted with ethyl acetate (150 ml), washed with water and brine, dried (Na₂SO₄) and evaporated in vacuo to give a yellow oil. The residue was purified by flash chromatography (eluant: 70% ethyl acetate, 30% pet. ether 60-80) to give a white solid identified as (2S)-[N^{α}-(tert-butyloxycarbonyl)-N^{ω}-(pyrazinyl-2-carbonyl)-L-ornithinyl]-4,4-difluoropyrrolidine-2-carbonitrile (148 mg, 0.33 mmol, 88%).

1G. (2S)-4,4-Difluoro-1-[N^{ω}-(pyrazinyl-2-carbonyl)-L-ornithinyl]pyrrolidine-2-carbonitrile Trifluoroacetate

(2S)-[N^{α}-(tert-Butyloxycarbonyl)-N^{ω}-(pyrazinyl-2-carbonyl)-L-ornithinyl]-4,4-difluoropyrrolidine-2-carbonitrile (135 mg, 0.3 mmol) was dissolved in trifluoroacetic acid (10 ml). The mixture was stirred for 1 hour at room temperature then the solvent was removed in vacuo to give a colourless oil identified as (2S)-4,4-difluoro-1-[N^{ω}-(pyrazinyl-2-carbonyl)-L-ornithinyl]-pyrrolidine-2-carbonitrile trifluoroacetate (140 mg, 0.3 mmol, 100%).

[M+H]⁺=353.1

¹H NMR (CD₃OD): δ 1.74-1.82 (2H,m), 1.90-2.02 (2H,m), 2.82-2.89 (2H,m), 3.30-3.32 (1H,m), 3.51 (2H,t,J=6.7 Hz), 4.12 (2H,t,J=11.9 Hz), 4.25-4.29 (1H,m), 4.88 (2H,s), 5.09-5.14 (1H,m), 8.67-8.68 (1H,m), 8.7 (1H,d,J=2.5 Hz), 9.23 (1H,d,J=1.4 Hz) ppm.

### Example 2

### 1-[N^{ω}-(5,6-Dichloronicotinoyl)-L-ornithinyl]-3,3-difluoropyrrolidine Hydrochloride

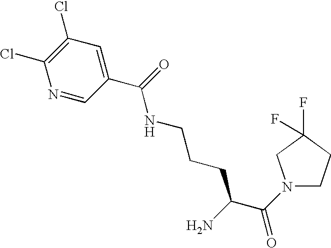

2A. 1-(tert-Butyloxycarbonyl)-3-pyrrolidone

(3R)-1-(tert-Butyloxycarbonyl)-3-hydroxypyrrolidine (980 mg, 5.3 mmol) was dissolved in CH₂Cl₂ (40 ml). Dess-Martin periodinane (2.5 g, 5.8 mmol) was added. The mixture was stirred for 3 hours at room temperature then the solvent was removed in vacuo and the residue was taken up in ethyl acetate (300 ml). The solution was washed with sat. NaHCO₃, water and brine, dried (Na₂SO₄) and evaporated in vacuo to give a colourless oil. The residue was purified by flash chromatography (eluant: 20% ethyl acetate, 80% pet. ether 60-80) to give a colourless oil identified as 1-(tert-butyloxycarbonyl)-3-pyrrolidone (842 mg, 4.6 mmol, 87%).

2B. 1(tert-Butyloxycarbonyl)-3,3-difluoropyrrolidine

1-(tert-Butyloxycarbonyl)-3-pyrrolidone (810 mg, 4.4 mmol) was dissolved in CH₂Cl₂ (30 ml). (Diethylamino)sulphur trifluoride (2.2 g, 13.7 mmol) was added to this solution at 0° C. The mixture was stirred for 18 hours at 0° C. to room temperature then carefully poured into sat. NaHCO₃ (100 ml). The mixture was stirred for 15 min then extracted with CH₂Cl₂. The organic extract was washed with water and brine, dried (Na₂SO₄) and evaporated in vacuo to give an orange oil. The residue was purified by flash chromatography (eluant: 10% ethyl acetate, 90% pet. ether 60-80) to give a colourless oil identified as 1-(tert-butyloxycarbonyl)-3,3-difluoropyrrolidine (580 mg, 2.8 mmol, 64%).

2C. 3,3-Difluoropyrrolidine Hydrochloride

1-(tert-Butyloxycarbonyl)-3,3-difluoropyrrolidine (540 mg, 2.6 mmol) was dissolved in 4M HCl/dioxan (30 ml). The solution was stirred for 1 hour at room temperature then the solvent was removed in vacuo to give an off white solid identified as 3,3-difluoropyrrolidine hydrochloride (370 mg, 2.6 mmol, 100%).

2D. N^{α}-(tert-Butyloxycarbonyl)-N^{ω}-(5,6-dichloronicotinoyl)-L-ornithine tert-butyl Ester

N^{α}-(tert-Butyloxycarbonyl)-L-ornithine tert-butyl ester hydrochloride (650 mg, 2.0 mmol) was dissolved in CH₂Cl₂/DMF (9:1, 40 ml). To this solution at 0° C. was added 5,6 dichloronicotinic acid (383 mg, 2.0 mmol), 1-hydroxybenzotriazole hydrate (459 mg, 3.0 mmol) and water-soluble carbodiimide (461 mg, 2.4 mmol). The mixture was stirred for 15 mins at 0° C. then the pH was adjusted to pH8 with N-methylmorpholine. The mixture was stirred for 18 h at 0° C. to room temperature then the solvent was removed in vacuo and the residue was taken up In ethyl acetate (100 ml). The solution was washed with 0.3M KHSO₄, sat. NaHCO₃, water and brine, dried (Na₂SO₄) and evaporated in vacuo to give a yellow oil. The residue was purified by flash chromatography (eluant: 50% ethyl acetate, 50% pet. ether 60-80) to give a white solid identified as N^{α}-(tert-butyloxycarbonyl)-N^{ω}-(5,6-dichloronicotinoyl)-L-ornithine tert-butyl ester (660 mg. 1.42 mmol, 71%).

2E. N^{α}-(tert-Butyloxycarbonyl)-N^{ω}-(5,6-dichloronicotinoyl)-L-ornithine

N^{α}-(tert-Butyloxycarbonyl)-N^{ω}-(5,6-dichloronicotinoyl)-L-ornithine tert-butyl ester (650 mg, 1.40 mmol) was dissolved in trifluoroacetic acid/dichloromethane (1:1, 20 ml). The mixture was stirred for 2 hours at room temperature then the solvent was removed in vacuo. The residue was dissolved in dioxan (20 ml) and aqueous potassium hydrogen carbonate (1M, 10 ml) and di-tert-butyl dicarbonate (327 mg, 1.5 mmol) were added. The mixture was stirred for 18 hours at room temperature then the dioxan was removed in vacuo. The residue was diluted with water, washed with. diethyl ether, acidified to pH2 with 1M HCl and extracted with chloroform. The organic extract was washed with water and brine, dried (Na₂SO₄) and evaporated in vacuo to give a colourless oil identified as N^{α}-(tert-butyloxycarbonyl)-N^{ω}-(5,6-dichloronicotinoyl)-L-ornithine (530 mg, 1.34 mmol, 96%).

2F. 1-[N^{α}-(tert-Butyloxycarbonyl)-N^{ω}-(5,6-dichloronicotinoyl)-L-ornithinyl]-3,3-difluoropyrrolidine

N^{α}-(tert-Butyloxycarbonyl)-N^{ω}-(5,6-dichloronicotinoyl)-L-ornithine (98 mg, 0.24 mmol) was dissolved in CH₂Cl₂ (20 ml). To this solution at 0° C. was added 3,3-difluoropyrrolidine hydrochloride (36 mg, 0.25 mmol), PyBOP (139 mg, 0.27 mmol) and triethylamine (60 mg, 0.6 mmol). The mixture was stirred for 18 h at 0° C. to room temperature then the solvent was removed in vacua and the residue was taken up in ethyl acetate (70 ml). The solution was washed with 0.3M KHSO₄, sat. NaHCO₃, water and brine, dried (Na₂SO₄) and evaporated in vacuo to give an orange oil. The residue was purified by flash chromatography (eluant: 60% ethyl acetate, 40% pet. ether 60-80) to give a colourless oil identified as 1-[N^{α}-(tert-butyloxycarbonyl)-N^{ω}-(5,6-dichloronicotinoyl)-L-ornithinyl]-3,3-difluoropyrrolidine (79 mg, 0.16 mmol, 68%).

2G. 1[N^{ω}-(5,6-Dichloronicotinoyl)-L-ornithinyl]-3,3-difluoropyrrolidine Hydrochloride

1-[N^{α}-(tert-Butyloxycarbonyl)-N^{ω}-(5,6-dichloronicotinoyl)-L-ornithinyl]-3,3-difluoropyrrolidine (68 mg, 0.14 mmol) was dissolved in 4M HCl/dioxan (20 ml). The mixture was stirred for 1 hour at room temperature then the solvent was removed in vacuo to give a colourless oil identified as 1-[N^{ω}-(5,6-dichloronicotinoyl)-L-ornithinyl]-3,3-difluoropyrrolidine hydrochloride (49 mg, 0.117 mmol, 83%).

[M+H]³⁰ =395.1

¹H NMR (CD₃OD): δ 1.28-1.34 (2H,m), 1.72-1.76 (2H,m), 1.85-1.92 (2H,m), 2.25-2.71 (2H,m), 3.30-3.41 (2H,m), 3.87-4.30 (6H,m), 8.36-8.39 (1H,m), 8.73-8.79 (1H,m) ppm.

### Example 3

### 3,3-Difluoro-1-[N^{ω}-(2-quinoxaloyl)-L-lysinyl]pyrrolidine Hydrochloride

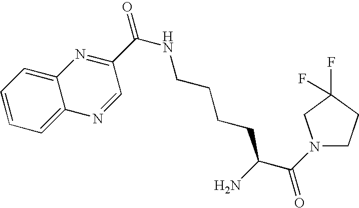

3A. N^{α}-(tert-Butyloxycarbonyl)-N^{ω}-(2-quinoxaloyl)-L-lysine Methyl Ester

N^{α}-(tert-Butyloxycarbonyl)-L-lysine methyl ester acetate (640 mg, 2.0 mmol) was dissolved in CH₂Cl₂ (40 ml). To this solution at 0° C. was added 2-quinoxaloyl chloride (385 mg, 2.0 mmol) and triethylamine (60 mg, 0.6 mmol). The mixture was stirred for 18 h at 0° C. to room temperature then the solvent was removed in vacuo and the residue was taken up in ethyl acetate (100 ml). The solution was washed with 0.3M KHSO₄, sat. NaHCO₃, water and brine, dried (Na₂SO₄) and evaporated in vacuo to give a yellow oil. The residue was purified by flash chromatography (eluant: 65% ethyl acetate, 35% pet. ether 60-80) to give a white solid identified as N^{α}-(tert-butyloxycarbonyl)-N^{ω}-(2-quinoxaloyl)-L-lysine methyl ester (580 mg, 1.40 mmol, 70%).

3 B. N^{α}-(tert-Butyloxycarbonyl)-N^{ω}-(2-quinoxaloyl)-L-lysine

N^{α}(tert-Butyloxycarbonyl)-N^{ω}-(2-quinoxaloyl)-L-lysine methyl ester (570 mg, 1.37 mmol) was dissolved in THF (50 ml). Aqueous lithium hydroxide (1M, 2 ml, 2.0 mmol) was added. The mixture was stirred for 3 hours at room temperature then the reaction mixture was diluted with ethyl acetate (150 ml), washed with 1M HCl, water and brine, dried (Na₂SO₄) and evaporated in vacuo to give a white solid identified as N^{α}-(tert-butyloxycarbonyl)-N^{ω}-(2-quinoxaloyl)-L-lysine (440 mg, 1.1 mmol, 80%).

3C. 1-[N^{α}-(tert-Butyloxycarbonyl)-N^{ω}-(2-quinoxaloyl)-L-lysinyl]-3,3-difluoropyrrolidine

N^{α}(tert-Butyloxycarbonyl)-N^{ω}-(2-quinoxaloyl)-L-lysine (95 mg, 0.24 mmol) was dissolved in CH₂Cl₂ (20 ml). To this solution at 0° C. was added 3,3-difluoropyrrolidine hydrochloride (34 mg, 0.24 mmol), PyBOP (145 mg, 0.28 mmol) and triethylamine (60 mg, 0.6 mmol). The mixture was stirred for 18 h at 0° C. to room temperature then the solvent was removed in vacuo and the residue was taken up in ethyl acetate (70 ml). The solution was washed with 0.3M KHSO₄, sat. NaHCO₃, water and brine, dried (Na₂SO₄) and evaporated in vacuo to give an orange oil. The residue was purified by flash chromatography (eluant: 60% ethyl acetate, 40% pet. ether 60-80) to give a colourless oil identified as 1-[N^{α}-(tert-butyloxycarbonyl)-N^{ω}-(2-quinoxaloyl)-L-lysinyl]-3,3-difluoropyrrolidine (87 mg, 0.18 mmol, 75%).

3D. 3,3-Difluoro-1-[N^{ω}-(2-quinoxaloyl)-L-lysinyl]pyrrolidine Hydrochloride

1-[N^{α}-(tert-Butyloxycarbonyl)-N^{ω}-(2-quinoxaloyl)-L-lysinyl]-3,3-difluoropyrrolidine (87 mg, 0.18 mmol) was dissolved in 4M HCl/dioxan (20 ml). The mixture was stirred for 1 hour at room temperature then the solvent was removed in vacuo to give a colourless oil identified as 3,3-difluoro-1-[N^{ω}-(2-quinoxaloyl)-L-lysinyl]pyrrolidine hydrochloride (75 mg, 0.18 mmol, 100%).

[M+H]⁺=392.3

¹H NMR (CD₃OD): δ 1.51-1.59 (2H,m), 1.70-1.78 (2H,m), 1.81-1.90 (2H,m), 2.37-2.58 (2H,m), 3.51-3.59 (2H,m), 3.62-4.32 (8H,m), 7.88-7.91 (2H,m), 8.10-8.21 (2H,m), 9.41 (1H,s) ppm.

### Example 4

### 3,3-Difluoro-1-[N^{ω}-(3-hydroxy-2-quinoxaloyl)-L-lysinyl]pyrrolidine Hydrochloride

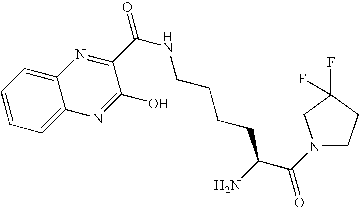

4A. 1-[N^{α}-(tert-Butyloxycarbonyl)-N^{ω}-(9-fluorenylmethyloxycarbonyl)-L-lysinyl]-3,3-difluoropyrrolidine

N^{α}-(tert-Butyloxycarbonyl)-N^{ω}-(9-fluorenylmethyloxycarbonyl)-L-lysine (1.14 g, 2.4 mmol) was dissolved in CH₂Cl₂/DMF (9:1, 100 ml). To this solution at 0° C. were added 1-hydroxybenzotriazole hydrate (394 mg, 2.9 mmol), water-soluble carbodiimide (680 mg, 3.4 mmol), 3,3-difluoropyrrolidine hydrochloride (380 mg, 2.43 mmol) and triethylamine (400 mg, 4 mmol). The mixture was stirred for 18 h at 0° C. to room temperature then the solvent was removed in vacuo and the residue was taken up in ethyl acetate (200 ml). The solution was washed with 0.3M KHSO₄, sat. NaHCO₃, water and brine, dried (Na₂SO₄) and evaporated in vacuo. The residue was purified by flash chromatography (eluant: 65% ethyl acetate, 35% pet. ether 60-80) to give a white solid identified as 1-[N^{α}-(tert-butyloxycarbonyl)-N^{ω}-(9-fluorenylmethyloxycarbonyl)-L-lysinyl]-3,3-difluoropyrrolidine (1.0 g, 1.8 mmol, 75%).

4B. 1-[N^{α}-(tert-Butyloxycarbonyl)-L-lysinyl]-3,3-difluoropyrrolidine

1-[N^{α}-(tert-Butyloxycarbonyl)-N^{ω}-(9-fluorenylmethyloxycarbonyl)-L-lysinyl]-3,3-difluoropyrrolidine (1.0 g, 1.8 mmol) was dissolved in THF (20 ml). Diethylamine (5 ml) was added. The mixture was stirred for 3 hours at room temperature then the solvent was removed in vacuo and the residue was purified by flash chromatography (eluant: 90% chloroform, 7% methanol, 3% triethylamine) to give a pale yellow oil identified as 1-[N^{α}-(tert-butyloxycarbonyl)-L-lysinyl]-3,3-difluoropyrrolidine (598 mg, 1.78 mmol, 99%).

4C. 1-[N^{α}-(tert-Butyloxycarbonyl)-N^{ω}-(3-hydroxy-2-quinoxaloyl)-L-lysinyl]-3,3-difluoropyrrolidine

1-[N^{α}-(tert-Butyloxycarbonyl)-L-lysinyl]-3,3-difluoropyrrolidine (147 mg, 0.44 mmol) was dissolved in CH₂Cl₂ (20 ml). To this solution at 0° C. was added 3-hydroxy-2-quinoxaline-carboxylic acid (83 mg, 0.44 mmol), PyBOP (274 mg, 0.53 mmol) and triethylamine (100 mg, 10 mmol). The mixture was stirred for 18 h at 0° C. to room temperature then the solvent was removed in vacuo and the residue was taken up in ethyl acetate (70 ml). The solution was washed with 0.3M KHSO₄, sat. NaHCO₃, water and brine, dried (Na₂SO₄) and evaporated in vacuo to give an orange oil. The residue was purified by flash chromatography (eluant: 96% dichloromethane, 4% methanol) to give a yellow gummy solid identified as 1-[N^{α}-(tert-butyloxycarbonyl)-N^{ω}-(3-hydroxy-2-quinoxaloyl)-L-lysinyl]-3,3-difluoropyrrolidine (106 mg, 0.21 mmol, 47%).

4D. 3,3-Difluoro-1-[N^{ω}-(3-hydroxy-2-quinoxaloyl)-L-lysinyl]pyrrolidine Hydrochloride

1-[N^{α}-(tert-Butyloxycarbonyl)-N^{ω}-(3-hydroxy-2-quinoxaloyl)-L-lysinyl]-3,3-difluoropyrrolidine (106 mg, 0.3 mmol) was dissolved in 4M HCl/dioxan (20 ml). The mixture was stirred for 1 hour at room temperature then the solvent was removed in vacuo to give a colourless oil identified as 3,3-difluoro-1-[N^{ω}-(3-hydroxy-2-quinoxaloyl)-L-lysinyl]-pyrrolidine hydrochloride (66 mg, 0.15 mmol, 50%).

[M+H]⁺=408.1

¹H NMR (CD₃OD): δ 1.85-1.87 (6H,m), 2.3-2.7 (2H,br m), 3.29-3.31 (6H,m), 3.4-3.7 (5H,br m), 7.35-7.5 (2H,m), 7.6-7.8 (1H,m), 7.9-8.0 (1H,m) ppm.

### Example 5

### 1-[N^{ω}-(3,4-Dichlorobenzyl)glutaminyl]-3,3-difluoropyrrolidine Hydrochloride

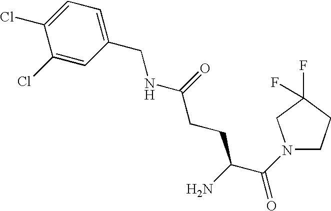

5A. 1-[N-(tert-Butyloxycarbonyl)-O^{ω}-methylglutamyl]-3,3-difluoropyrrolidine

N-(tert-Butyloxycarbonyl)-O^{ω}-methylglutamic acid (462 mg, 1.04 mmol) was dissolved in CH₂Cl₂/DMF (9:1, 20 ml). To this solution at 0° C. were added 1-hydroxybenzotriazole hydrate (192 mg, 1.25 mmol), water-soluble carbodiimide (277 mg, 1.46 mmol), 3,3-difluoropyrrolidine hydrochloride (150 mg, 1.04 mmol) and triethylamine (200 mg, 2.0 mmol). The mixture was stirred for 18 h at 0° C. to room temperature then the solvent was removed in vacuo and the residue was taken up in ethyl acetate (70 mL). The solution was washed with 0.3M KHSO₄, sat. NaHCO₃, water and brine, dried (Na₂SO₄) and evaporated in vacuo. The residue was purified by flash chromatography (eluant: 40% ethyl acetate, 60% pet. ether 60-80) to give a colourless oil identified as 1-[N-(tert-butyloxycarbonyl)-O^{ω}-methylglutamyl]-3,3-difluoropyrrolidine (362 mg, 1.03 mmol, 99%).

5B. 1-[N-(tert-Butyloxycarbonyl)glutamyl]-3,3-difluoropyrrolidine

1-[N-(tert-Butyloxycarbonyl)-O^{ω}-methylglutamyl]-3,3-difluoropyrrolidine (362 mg, 1.03 mmol) was dissolved in dioxan (5 ml). Aqueous lithium hydroxide (1M, 2:5 ml, 2.5 mmol) was added. The mixture was stirred for 1 hour at room temperature then the solvent was removed in vacuo and the residue was taken up in ethyl acetate (70 mL). The solution was washed with 1M KHSO₄, water and brine, dried (Na₂SO₄) and evaporated in vacuo to give a colourless oil identified as 1-[N-(tert-butyloxycarbonyl)glutamyl]-3,3-difluoropyrrolidine (200 mg, 0.66 mmol, 58%).

5C. 1-[N^{α}-(tert-Butyloxycarbonyl)-N^{ω}-(3,4-dichlorobenzyl)glutaminyl]-3,3-difluoropyrrolidine

1-[N-(tert-Butyloxycarbonyl)glutamyl]-3,3-difluoropyrrolidine (100 mg, 0.30 mmol) was dissolved in CH₂Cl₂/DMF (9:1, 20 ml). To this solution at 0° C. were added 1-hydroxybenzotriazole hydrate (53 mg, 0.36 mmol), water-soluble carbodiimide (80 mg, 0.42 mmol), 3,4-dichlorobenzylamine (53 mg, 0.4 mmol) and triethylamine (61 mg, 0.6 mmol). The mixture was stirred for 18 h at 0° C. to room temperature then the solvent was removed in vacuo and the residue was taken up in ethyl acetate (200 ml). The solution was washed with 0.3M KHSO₄, sat. NaHCO₃, water and brine, dried (Na₂SO₄) and evaporated in vacuo. The residue was purified by flash chromatography (eluant: 75% ethyl acetate, 25% pet. ether 60-80) to give a white solid identified as 1-[N^{α}-(tert-butyloxycarbonyl)-N^{ω}-(3,4-dichlorobenzyl)glutaminyl]-3,3-difluoropyrrolidine (144 mg, 0.29 mmol, 100%).

5D. 1-[N^{ω}-(3,4-Dichlorobenzyl)glutaminyl]-3,3-difluoropyrrolidine Hydrochloride

1-[N^{α}-(tert-Butyloxycarbonyl)-N^{ω}-(3,4-dichlorobenzyl)glutaminyl]-3,3-difluoropyrrolidine (144 mg, 0.29 mmol) was dissolved in 4M HCl/dioxan (20 ml). The mixture was stirred for 1 hour at room temperature then the solvent was removed in vacuo to give a white solid identified as 1-[N^{α}-(3,4-dichlorobenzyl)glutaminyl]-3,3-difluoropyrrolidine hydrochloride (120 mg, 0.28 mmol, 100%).

[M+H]⁺=394.0, 395.7

¹H NMR (CD₃OD): δ 2.00-2.20 (2H,m), 2.30-2.50 (4H,m), 3.25-3.35 (3H,m), 3.60-4.20 (4H,m), 4.20-4.40 (3H,m), 7.20-7.30 (1H,m), 7.40-7.50 (2H,m) ppm

### Example 6

### (3S)-3-Fluoro-1-[N^{ω}-(2-quinoxaloyl)-L-lysinyl]pyrrolidine Hydrochloride

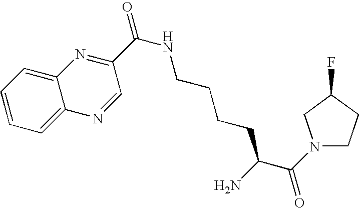

6A. (3S)-1-(tert-Butyloxycarbonyl)-3-fluoropyrrolidine

(3R)-N-(tert-Butyloxycarbonyl)-3-hydroxypyrrolidine (1.0 g, 5.34 mmol) was dissolved in CH₂Cl₂ (30 ml). (Diethylamino)sulphur trifluoride (860 g, 5.34 mmol) was added to this solution at −78° C. The mixture was stirred for 18 hours at −78° C. to room temperature then the reaction mixture was carefully poured into sat. NaHCO₃ (100 ml) and stirred for 15 min and extracted with CH₂Cl₂. The organic extract was washed with water and brine, dried (Na₂SO₄) and evaporated in vacuo to give an orange oil. The residue was purified by flash chromatography (eluant: 28% ethyl acetate, 72% pet. ether 60-80) to give a colourless oil identified as (3S)-1-(tert-butyloxycarbonyl)-3-fluoropyrrolidine (507 mg, 2.67 mmol, 50%).

6B. (3S)-3-Fluoropyrrolidine Hydrochloride

(3S)-1-(tert-Butyloxycarbonyl)-3-fluoropyrrolidine (507 mg, 2.68 mmol) was dissolved in 4M HCl/dioxan (30 ml). The mixture was stirred for 1 hour at room temperature then the solvent was removed in vacuo to give an off-white solid identified as (3S)-3-fluoropyrrolidine hydrochloride (320 mg, 2.6 mmol, 95%).

6C. (3S)-1-[N^{α}-(tert-Butyloxycarbonyl)-N^{ω}-(2-quinoxaloyl)-L-lysinyl]-3-fluoropyrrolidine

N^{α}-(tert-Butyloxycarbonyl)-N^{ω}-(2-quinoxaloyl)-L-lysine (50 mg, 0.124 mmol) was dissolved in CH₂Cl₂ (20 ml). To this solution at 0° C. was added (3S)-3-fluoropyrrolidine hydrochloride (17 mg, 0.136 mmol), 1-hydroxybenzotriazole hydrate (20 mg, 0.149 mmol), water-soluble carbodiimide (35 mg, 0.17 mmol) and triethylamine (30 mg, 0.3 mmol). The mixture was stirred for 18 h at 0° C. to room temperature then the solvent was removed in vacuo and the residue was taken up in ethyl acetate (70 ml). The solution was washed with 0.3M KHSO₄, sat. NaHCO₃, water and brine, dried (Na₂SO₄) and evaporated in vacuo to give an orange oil. The residue was purified by flash chromatography (eluant: 60% ethyl acetate, 40% pet. ether 60-80) to give a colourless oil identified as (3S)-1-[N^{α}-(tert-butyloxycarbonyl)-N^{ω}-(2-quinoxaloyl)-L-lysinyl]-3-fluoropyrrolidine (50 mg, 0.107 mmol, 86%).

6D. (3S)-3-Fluoro-1-[N^{ω}-(2-quinoxaloyl)-L-lysinyl]pyrrolidine Hydrochloride

(3S)-1-[N^{α}-(tert-Butyloxycarbonyl)-N^{ω}-(2-quinoxaloyl)-L-lysinyl]-3-fluoropyrrolidine (50 mg, 0.105 mmol) was dissolved in 4M HCl/dioxan (10 ml). The mixture was stirred for 1 hour at room temperature then the solvent was removed in vacuo to give an off-white solid identified as (3S)-3-fluoro-1-[N^{ω}-(2-quinoxaloyl)-L-lysinyl]pyrrolidine hydrochloride (43 mg, 0.105 mmol, 100%).

[M+H]⁺=374.0

¹H NMR (CD₃OD): δ 1.53-1.57 (2H,m), 1.72-1.75 (2H,m), 1.92-1.94 (2H,m), 2.21-2.31 (1H,m), 3.43-4.01 (8H,m), 4.16-4.18 (1H,m), 5.19-5.39 (1H,m), 7.96-7.97 (2H,m), 8.16-8.21 (2H,m), 9.41(1H,s) ppm.

### Example 7

### (2S)-1-[N^{α}-(1′-Acetoxyethoxycarbonyl)-N^{ω}-(pyrazinyl-2-carbonyl)-L-ornithinyl]-4,4-difluoropyrrolidine-2-carbonitrile

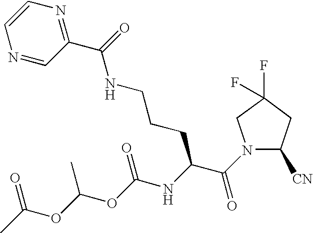

A solution of (2S)-1-[N^{ω}-(pyrazinyl-2-carbonyl)-L-ornithinyl]-4,4-difluoropyrrolidine-2-carbonitrile trifluoroacetate (40 mg, 0.086 mmol), α-acetoxyethyl p-nitrophenyl carbonate (28 mg, 0.11 mmol; prepared according to Alexander et al., J. Med. Chem. 31, 318, 1988) and triethylamine (20 mg, 0.2 mmol) in dichloromethane (25 ml) was stirred at room temperature for 18 hours, then evaporated in vacuo. The residue taken up in ethyl acetate (70 ml). The solution was washed with sat NaHCO₃, water and brine, dried (Na₂SO₄) and evaporated. The residue was purified by flash chromatography (eluant 98% chloroform, 2%methanol) to give a white solid identified as (2S)-1-[N^{α}-(1′-acetoxyethoxycarbonyl)-N^{ω}-(pyrazinyl-2-carbonyl)-L-ornithinyl]pyrrolidine-2-carbonitrile (26 mg, 0.053 mmol, 62%).

[M+H]⁺=483.1

¹H NMR (CDCl₃): δ 1.41-1.46 (3H,m), 1.72-1.83 (4H,m), 2.01-2.05 (3H,m), 2.68-2.74 (2H,m), 3.49-3.58 (2H,m), 4.03-4.11 (2H,m), 4.41-4.43 (1H,m), 4.94-4.98 (1H,m), 5.56 (1H,d,J=8.6 Hz), 6.73-6.76 (1H,m), 7.90-7.93 (1H,m), 8.51-8.52 (1H,m), 8.75 (1H,d,J=2.4 Hz), 9.37 (1H,d,J=1.4 Hz) ppm.

### Example 8

### 1-[N^{α}-(Acetoxymethoxycarbonyl)-N^{ω}-(5,6-dichloronicotinoyl)-L-ornithinyl]-3,3-difluoropyrrolidine

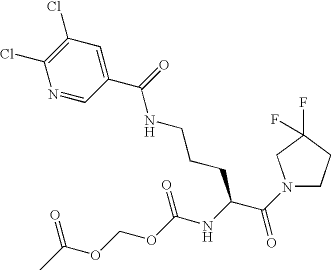

1-[N^{α}-(tert-Butyloxycarbonyl)-N^{ω}-(5,6-dichloronicotinoyl)-L-ornithinyl]-3,3-difluoropyrrolidine (88 mg, 0.18 mmol) was dissolved in 4M HCl/dioxan (20 ml). The mixture was stirred for 1 hour at room temperature then the solvent was removed in vacuo. The residue was dissolved in dichloromethane (25 ml), acetoxymethyl p-nitrophenyl carbonate (60 Mg, 0.24 mmol; prepared according to Alexander et al., J. Med. Chem. 31, 318, 1988) and triethylamine (60 mg, 0.6 mmol) were added, and the mixture was stirred at room temperature for 18 hours. The solution was evaporated in vacuo and the residue was taken up in ethyl acetate (70 ml). The solution was washed with sat NaHCO₃, water and brine, dried (Na₂SO₄) and evaporated in vacuo. The residue was purified by flash chromatography (eluant 80% ethyl acetate, 20% pet. Ether 60-80) to give a white solid identified as 1-[N^{α}-acetoxymethoxycarbonyl-N^{ω}-(5,6-dichloronicotinoyl)-L-ornithinyl]-3,3-difluoropyrrolidine (64 mg, 0.126 mmol, 71%).

[M+H]⁺=512.8

¹H NMR (CDCl₃): δ 1.66-1.78 (4H,m), 2.01 (3H,s), 2.36-2.67 (2H,m), 3.49-3.53 (2H,m), 3.63-3.87 (4H,m), 4.25-4.70 (1H,m), 5.62-5.65 (1H,m), 5.72-5.76 (1H,m), 5.97-6.01 (1H,m), 6.85-7.09 (1H,m), 8.26 (1H,d,J=2 Hz), 8.61 (1H,d,J=2.2 Hz) ppm.

The following compounds were prepared using analogous methods.

### Examples 9-22

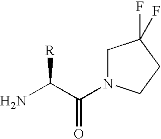

| Ex No | R |
|---|---|
| 9 | Isopropyl |
| 10 | n-Butyl |
| 11 | sec-Butyl |
| 12 | tert-Butyl |
| 13 | Cyclohexyl |
| 14 | Benzyl |
| 15 | 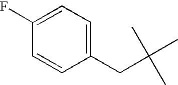 |
| 16 | CH₃S(CH₂)₂ |
| 17 | HOCH₂ |
| 18 | 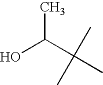 |
| 19 | 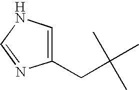 |
| 20 | HO₂CCH₂ |
| 21 | 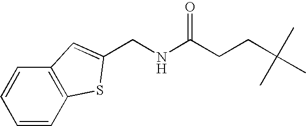 |
| 22 | 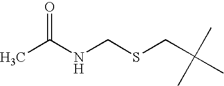 |

### Examples 23-29

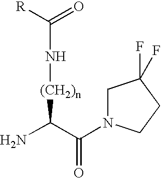

| Ex No | n | R |
|---|---|---|
| 23 24 | 3 4 | 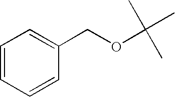 |
| 25 | 4 | NH₂ |
| 26 27 | 3 4 | 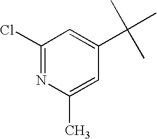 |
| 28 | 3 | 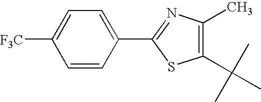 |
| 29 | 3 | 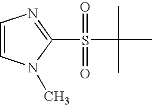 |

### Example 30-36

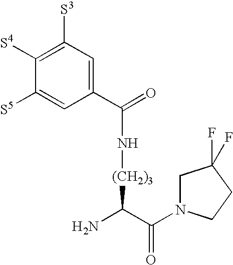

| Ex No | S³ | S⁴ | S⁵ |
|---|---|---|---|
| 31 | CN | H | H |
| 32 | NO₂ | H | H |
| 33 | Cl | H | Cl |
| 34 | H | Cl | H |
| 35 | Cl | H | H |
| 36 | CH₃ | H | H |

### Example 37-61

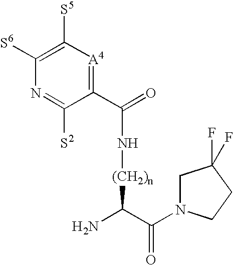

| Ex No | n | S² | A⁴ | S⁵ | S⁶ |
|---|---|---|---|---|---|
| 37 | 3 | H | CH | H | Cl |
| 38 | 3 | H | CH | H | CH₃ |
| 39 | 3 | H | CH | H | CF₃ |
| 40 | 3 | Cl | CH | H | Cl |
| 41 | 3 | Cl | CH | H | CH₃ |
| 42 | 3 | CH₃ | CH | H | CF₃ |
| 43 | 3 | H | N | —CH═CH—CH═CH— | |
| 44 | 3 | H | N | H | CH₃ |
| 45 | 3 | H | CH | —CH═CH—CH═CH— | |
| 46 | 3 | H | CH | Br | H |
| 47 | 3 | H | CH | H | SH |
| 48 | 3 | H | CH | H | CN |
| 49 | 3 | OH | N | —CH═CH—CH═CH— | |
| 50 | 3 | Cl | CH | H | H |
| 51 | 4 | CO₂H | CH | H | H |
| 52 | 4 | H | CH | Cl | OH |
| 53 | 4 | H | C(Cl) | —C(CH₃)═N—N(CH₃)— | |
| 54 | 4 | H | CH | Cl | Cl |
| 55 | 4 | H | CH | —CH═CH—CH═CH— | |
| 56 | 4 | H | CH | Br | H |
| 57 | 4 | H | CH | CH₃ | H |
| 58 | 4 | H | CH | H | SH |
| 59 | 4 | H | CH | H | CN |
| 60 | 4 | H | CH | H | CF₃ |
| 61 | 4 | H | N | H | CH₃ |

### Examples 62-84

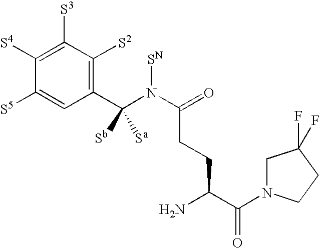

| Ex | | | | | | | |
|---|---|---|---|---|---|---|---|
| No | S^{a} | S^{b} | S^{N} | S² | S³ | S⁴ | S⁵ |
| 62 | H | H | H | Cl | H | H | H |
| 63 | H | H | H | H | F | H | H |
| 64 | H | H | H | H | CF₃ | H | H |
| 65 | H | H | H | H | H | F | H |
| 66 | H | H | H | H | H | Cl | H |
| 67 | H | H | H | H | CF₃ | H | CF₃ |
| 68 | H | H | H | H | Br | H | H |
| 69 | H | H | H | H | I | H | H |
| 70 | H | H | H | H | NO₂ | H | H |
| 71 | H | H | H | H | H | NO₂ | H |
| 72 | H | H | H | H | Cl | H | H |
| 73 | H | H | H | H | Cl | F | H |
| 74 | H | H | H | H | H | CH₃SO₂ | H |
| 75 | H | H | —CH₂—CH₂— | | H | H | H |
| 76 | H | H | H | CH₃SO₂ | H | H | H |
| 77 | H | H | H | CH₃SO₂NHCO | H | H | H |
| 78 | H | H | H | H | H₂NCO | H | H |
| 79 | H | H | H | —CH═CH—CH═CH— | | H | H |
| 80 | CH₃ | H | H | H | H | H | H |
| 81 | H | CH₃ | H | H | H | H | H |
| 82 | H | H | H | H | Cl | H | Cl |
| 83 | H | H | H | H | CH₃CO | H | H |
| 84 | H | H | H | H | CH₃ | H | H |

### Example 85-100

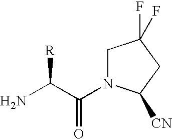

| Ex No | R |
|---|---|
| 85 | Isopropyl |
| 86 | n-Butyl |
| 87 | sec-Butyl |
| 88 | tert-Butyl |
| 89 | Cyclohexyl |
| 90 | Benzyl |
| 91 | 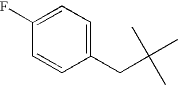 |
| 92 | CH₃S(CH₂)₂ |
| 93 | HOCH₂ |
| 94 | 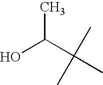 |
| 95 | 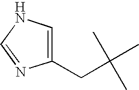 |
| 96 | HO₂CCH₂ |
| 97 | 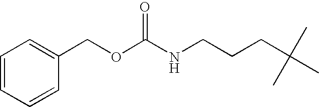 |
| 98 | 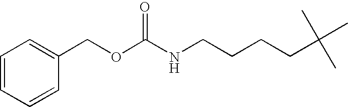 |
| 99 | 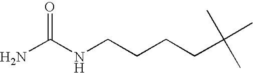 |
| 100 | 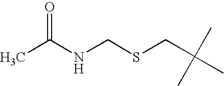 |

### Examples 101-126

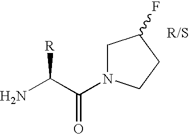

| Ex No | R/S | R |
|---|---|---|
| 101 | R | Isopropyl |
| 102 | S | |
| 103 | R | n-Butyl |
| 104 | S | |
| 105 | R | sec-Butyl |
| 106 | S | |
| 107 | R | tert-Butyl |
| 108 | S | |
| 109 | R | Cyclohexyl |
| 110 | S | |
| 111 | R | Benzyl |
| 112 | S | |
| 113 114 | R S | 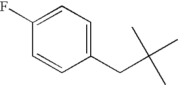 |
| 115 | R | CH₃S(CH₂)₂ |
| 116 | S | |
| 117 | R | HOCH₂ |
| 118 | S | |
| 119 120 | R S | 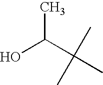 |
| 121 122 | R S | 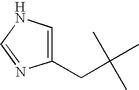 |
| 123 | R | HO₂CCH₂ |
| 124 | S | |
| 125 126 | R S | 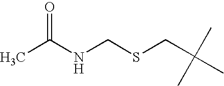 |

### Example 127-134

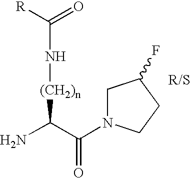

| Ex No | R/S | n | R |
|---|---|---|---|
| 127 128 129 130 | R R S S | 3 4 3 4 | 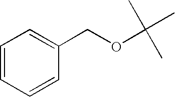 |
| 131 | R | 4 | NH₂ |
| 132 | S | | |
| 133 134 | R S | 3 | 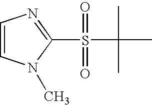 |

### Example 135-139

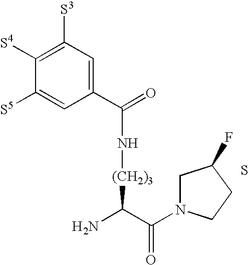

| Ex No | S³ | S⁴ | S⁵ |
|---|---|---|---|
| 135 | CN | H | H |
| 136 | NO₂ | H | H |
| 137 | Cl | H | Cl |
| 138 | H | Cl | H |
| 139 | Cl | H | H |

### Example 140-164

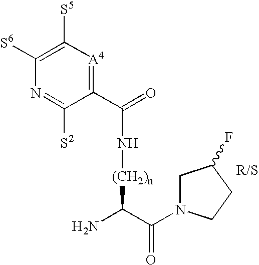

| Ex No | R/S | n | S² | A⁴ | S⁵ | S⁶ |
|---|---|---|---|---|---|---|
| 140 | S | 3 | H | CH | H | Cl |
| 141 | S | 3 | OH | CH | H | CH₃ |
| 142 | S | 3 | H | CH | H | OH |
| 143 | S | 3 | H | CH | H | CH₃ |
| 144 | S | 3 | H | CH | Cl | OH |
| 145 | S | 3 | H | C(Cl) | —C(CH₃)═N—N(CH₃)— | |
| 146 | S | 3 | H | CH | Cl | Cl |
| 147 | R | 3 | H | CH | Cl | Cl |
| 148 | S | 3 | Cl | CH | H | Cl |
| 149 | S | 3 | Cl | CH | H | CH₃ |
| 150 | S | 3 | H | N | —CH═CH—CH═CH— | |
| 151 | S | 3 | H | N | H | CH₃ |
| 152 | S | 3 | OH | N | —CH═CH—CH═CH— | |
| 153 | S | 3 | Cl | CH | H | H |
| 154 | S | 4 | CO₂H | OH | H | H |
| 155 | S | 4 | H | CH | Cl | OH |
| 156 | S | 4 | H | C(Cl) | —C(CH₃)═N—N(CH₃)— | |
| 157 | S | 4 | H | CH | Cl | Cl |
| 158 | S | 4 | H | CH | —CH═CH—CH═CH— | |
| 159 | S | 4 | H | CH | Br | H |
| 160 | S | 4 | H | CH | Cl | OH |
| 161 | S | 4 | OH | CH | —CH═CH—CH═CH— | |
| 162 | S | 4 | H | CH | CH₃ | H |
| 163 | S | 4 | H | CH | H | SH |

| | | | | | |
|---|---|---|---|---|---|
| 164 | R | 4 | H | N | —CH═CH—CH═CH— |

### Examples 165-166

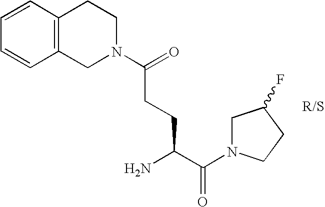

| Ex No | R/S |
|---|---|
| 165 | R |
| 166 | S |

### Example 167

### Determination of Activity

Compounds were assayed as inhibitors of DP-IV according to the methods described in WO95/15309. All the compounds described in the foregoing Examples were competitive inhibitors of DP-IV with Kᵢ values less than 300 nM, except for the compounds of Examples 7 and 8. These two compounds are prodrugs and do not show significant inhibition of DP-IV at concentrations up to 5 μM.

### Example 168

### Determination of Activity In Vivo

The anti-diabetic action of selected compounds was demonstrated in Zucker obese rats using a standard oral glucose tolerance test. Control rats were given a solution of glucose by oral gavage, and plasma glucose levels were determined. These rats demonstrated a significant hyperglycaemia. Compounds according to the present invention were dissolved in glucose solution at various concentrations, such that the rats could be given varying doses of the compound simultaneously with the glucose challenge. The hyperglycaemic excursion was reduced in a dose-dependent manner in animals receiving between 0.1 and 100 mg/kg of DP-IV inhibitor.

### Example 169

### Pharmaceutical Formulation

Tablets containing 100 mg of the compound of Example 1 as the active agent are prepared from the following:

| | |
|---|---|
| Compound or Example 1 | 200.0 g |
| Corn starch | 71.0 g |
| Hydroxypropylcellulose | 18.0 g |
| Carboxymethylcellulose calcium | 13.0 g |
| Magnesium stearate | 3.0 g |
| Lactose | 195.0 g |
| Total | 500.0 g |

The materials are blended and then pressed to give 2000 tablets of 250 mg, each containing 100 mg of the compound of Example 1.

The above demonstrates that the compounds according to the present invention are inhibitors of DP-IV or prodrugs thereof and would accordingly be expected to be useful as therapeutic agents for the treatment of impaired glucose tolerance, type II diabetes, and other diseases where inhibition of this enzyme leads to an improvement in the underlying pathology or the symptoms.

The present invention is further defined in the following Claims.

1. A compound according to general formula 1, or a pharmaceutically acceptable salt thereof, 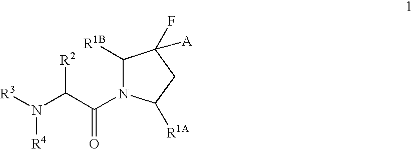
wherein:
A is F or H;
one of R^{1A} and R^{1B} is selected from H and CN and the other is H;
R² is selected from H, C₁-C₈ alkyl, optionally substituted phenyl, optionally substituted benzyl and R⁵; and
R³ is selected from H, C₁-C₈ alkyl, adamantyl, adamantylmethyl, adamantylethyl and Het-NH(CH₂)ₐ; or
R² and R³ together constitute a chain of three or four methylene groups so as to form, together with the atoms to which they are attached, a pyrrolidine or piperidine ring, which ring may further be fused with a benzenoid ring;
R⁴ is selected from H, R⁶OCO, H₂NCH(R⁷)CO, H₂NCH(R⁸)CONHCH(R⁹)CO, and a group according to general formula 2; 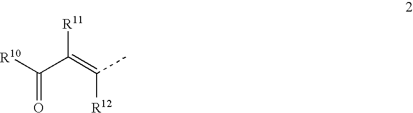
R⁵ is selected from CH₂R¹³, CH₂CH₂R¹³ and C(R¹⁴)(R¹⁵)—X¹—R¹⁶;
R⁶ is selected from C₁-C₆ alkyl, optionally substituted phenyl, optionally substituted benzyl and R¹⁷CO₂C(R¹⁸)(R¹⁹);
R⁷, R⁸ and R⁹ are each independently selected from the side chains of the proteinaceous amino acids;
R¹⁰ is selected from C₁-C₈ alkyl, phenyl and O—(C₁-C₈ alkyl);
R¹¹ is selected from H and C₁-C₈ alkyl;
R¹² is selected from H, C₁-C₈ alkyl and phenyl;
R¹³ is selected from CO—N(R²⁰)(R²¹), N(R²²)—C(═X²)R²³ and N(R²²)(R²⁴);
R¹⁴ and R¹⁵ are independently selected from H and methyl, or together are —(CH₂)_{z}—;
R¹⁶ is selected from C₁-C₈ alkyl, optionally substituted phenyl, optionally substituted benzyl and —(CH₂)_{b}—R¹³;
R¹⁷ is selected from H and C₁-C₈ alkyl;
R¹⁸ and R¹⁹ are independently selected from H and C₁-C₈ alkyl, or together are —(CH₂)_{y}—;
R²⁰ and R²¹ are independently selected from H, C₁-C₈ alkyl, optionally substituted phenyl, optionally substituted phenylalkyl, Het and —(CH₂)_{c}Het, or R²⁰ and R²¹ together constitute a chain of four or five methylene groups so as to form, together with the nitrogen atom to which they are attached, a pyrrolidine or piperidine ring, which ring may further be fused with a benzenoid ring;
R²² is selected from H and methyl;
R²³ is selected from R²⁵, O—R²⁵ and N(R²⁶)(R²⁷);
R²⁴ is selected from optionally substituted phenyl, Het and —CH₂-Het;
R²⁵ is selected from C₁-C₈ alkyl, optionally substituted phenyl, optionally substituted phenylalkyl, Het and —(CH₂)_{c}Het;
R²⁶ and R²⁷ are independently selected from H, C₁-C₈ alkyl, optionally substituted phenyl, optionally substituted phenylalkyl, Het and —(CH₂)_{c}Het, or R²⁶ and R²⁷ together constitute a chain of four or five methylene groups so as to form, together with the nitrogen atom to which they are attached, a pyrrolidine or piperidine ring, which ring may further be fused with a benzenoid ring;
Het is an aromatic nitrogen-containing heterocycle selected from pyridyl, pyridazinyl, pyrimidinyl, pyrazinyl, imidazolyl, pyrazolyl, thiazolyl, isothiazolyl, oxazolyl, isoxazolyl and benz-fused analogues of these, all of which may optionally be substituted on one or more carbon atoms, and where the substituents are selected from lower alkyl, hydroxy, lower alkyloxy, amino, lower alkylamino, di(lower alkyl)amino, fluoro, chloro, bromo, trifluoromethyl, nitro, cyano, carboxy and lower alkyloxycarbonyl groups;
X¹ is selected from —O—, —S— and —CH₂—;
X² is selected from O and S;
a is 2 or 3;
b is 1, 2 or 3;
c is 1 or 2; and
y and z are 2, 3 or 4.

2. A compound according to claim 1, or a pharmaceutically acceptable salt thereof, wherein R^{1A} and R^{1B} are both H.

3. A compound according to claim 1, or a pharmaceutically acceptable salt thereof, wherein R^{1A} is CN and R^{1B} is H.

4. A compound according to claim 1, or a pharmaceutically acceptable salt thereof, wherein R^{1A} is H and R^{1B} is CN.

5. A compound according to claim 1, or a pharmaceutically acceptable salt thereof, wherein A is F.

6. A compound according to claim 1, or a pharmaceutically acceptable salt thereof, wherein A is H.

7. A compound according to claim 1, or a pharmaceutically acceptable salt thereof, wherein R⁴ is H.

8. A compound according to claim 1, or a pharmaceutically acceptable salt thereof, wherein R³ is H.

9. A compound according to claim 1, or a pharmaceutically acceptable salt thereof, wherein R² is H and R³ is selected from adamantyl, adamantylmethyl, adamantylethyl and Het-NH(CH₂)ₐ.

10. A compound according to claim 9, or a pharmaceutically acceptable salt thereof, wherein R³ is Het-NH(CH₂)ₐ.

11. A compound according to claim 10, or a pharmaceutically acceptable salt thereof, wherein a is 2 and Het is 5-substituted-2-pyridyl.

12. A compound according to claim 1, or a pharmaceutically acceptable salt thereof, wherein R³ is H and R² is selected from C₁-C₈ alkyl, optionally substituted phenyl, optionally substituted benzyl and R⁵.

13. A compound according to claim 12, or a pharmaceutically acceptable salt thereof, wherein R² is C₁-C₈ alkyl.

14. A compound according to claim 12, or a pharmaceutically acceptable salt thereof, wherein R² is R⁵.

15. A compound according to claim 14, or a pharmaceutically acceptable salt thereof, wherein R⁵ is selected from CH₂CH₂R¹³ and C(R¹⁴)(R¹⁵)—X¹—R¹⁶.

16. A compound according to claim 15, or a pharmaceutically acceptable salt thereof, wherein R⁵ is CH₂CH₂R¹³ and R¹³ is CO—N(R²⁰)(R²¹).

17. A compound according to claim 15, or a pharmaceutically acceptable salt thereof, wherein R⁵ is C(R¹⁴)(R¹⁵)—X¹—R¹⁶, R¹⁴ and R¹⁵ are independently selected from H and methyl, and R¹⁶ is —(CH₂)_{b}—R¹³.

18. A compound according to claim 17, or a pharmaceutically acceptable salt thereof, wherein R¹⁴ and R¹⁵ are both H, X¹ is CH₂ and b is 1 or 2.

19. A compound according to claim 18, or a pharmaceutically acceptable salt thereof, wherein R¹³ is selected from N(R²²)—C(═X²)R²³ and N(R²²)(R²⁴).

20. A compound according to claim 19, or a pharmaceutically acceptable salt thereof, wherein R¹³ is N(R²²)—C(═X²)R²³, R²² is H and X² is O.

21. A compound according to claim 20, or a pharmaceutically acceptable salt thereof, wherein R²³ is Het.

22. A compound according to claim 1 wherein R² is other than H and the absolute stereochemistry is as shown in general formula 3.

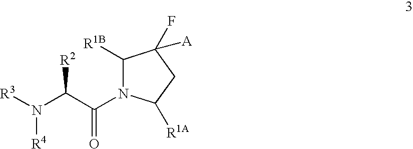

23. A compound according to claim 1 wherein R^{1A} is CN, R^{1B} is H and the absolute stereochemistry is as shown in general formula 4.

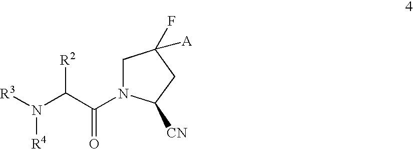

24. A compound according to claim 1 wherein R^{1A} is H, R^{1B} is CN and the absolute stereochemistry is as shown in general formula 5.

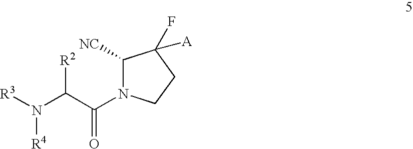

25. A pharmaceutical composition for human therapeutic use comprising at least one compound according to claim 1, or a pharmaceutically acceptable salt thereof.

26. A composition according to claim 25 for the treatment of type 2 diabetes or impaired glucose tolerance.

27. A composition according to claim 25 for the treatment of growth hormone deficiency or polycystic ovary syndrome.

28. A composition according to claim 25 for the treatment of auto-immune and inflammatory diseases.

29. The use of a compound according to claim 1, or a pharmaceutically acceptable salt thereof, for the preparation of a pharmaceutical composition for the treatment of type 2 diabetes, impaired glucose tolerance, growth hormone deficiency, polycystic ovary syndrome, and auto-immune and inflammatory diseases.

30. The use of a compound according to claim 1, or a pharmaceutically acceptable salt thereof, for the treatment of type 2 diabetes, impaired glucose tolerance, growth hormone deficiency, polycystic ovary syndrome, and auto-immune and inflammatory diseases.

31. A method of treatment for type 2 diabetes, impaired glucose tolerance, growth hormone deficiency, polycystic ovary syndrome, and auto-immune and inflammatory diseases, which comprises the administration to a person in need of such treatment of a therapeutically effective amount of a compound according to claim 1 or a pharmaceutically acceptable salt thereof.

32. At least one optical isomer of a compound according to claim 1.

33. A tautomer of a compound according to claim 1.
